(19) United States
(12) Patent Application Publication
(10) Pub. No.: US 2007/0202223 A1
(43) Pub. Date: Aug. 30, 2007

(54) Egg Quality Measurement

(75) Inventor: Eli Oren, Or-Yehuda (IL)

Correspondence Address: WOLF, BLOCK, SCHORR & SOLIS-COHEN LLP, 250 PARK AVENUE, NEW YORK, NY, 10177, US

(73) Assignee: Orka Food Technology Ltd., Ramat-Hasharon (IL)

(21) Appl. No.: 11/463,367

(22) Filed: Aug. 9, 2006

Related U.S. Application Data
(60) Provisional application No. 60/720,038, filed on Sep. 26, 2005.

(51) Int. Cl. G01N 33/02

(52) U.S. Cl. 426/231; 426/614

(57) ABSTRACT

A method of determining an effective height of albumen of an egg, for calculating Haugh units of the egg, the method comprising: a) breaking the egg and placing its contents on a surface; b) choosing at least one location at which to measure the height of the albumen; and c) measuring the height of the albumen of the egg above the surface at the at least one location, using a non-contact measurement method.

### RELATED APPLICATIONS

This application claims priority from U.S. provisional application 60/720,038, entitled “Method and apparatus for evaluating, measuring and displaying an egg quality and Yolk color automatically,” filed on Sep. 26, 2005, the disclosure of which is incorporated herein by reference.

### FIELD OF THE INVENTION

The field of the invention is measurement of the quality of eggs.

### BACKGROUND OF THE INVENTION

Qualitative and quantitative measurements of egg quality may be useful for complying with legal requirements on the quality of eggs that may be offered for sale, and as a marketing technique to reassure customers of the high quality of the eggs that they are buying. Such measurements include both exterior and interior measurements.

Exterior measurements (i.e. measurements made without breaking or piercing the shell) have traditionally been done by visual inspection of the outer surface of an intact egg, to search for cracks, and by candling. More recently, U.S. Pat. No. 5,615,777 to Weichman describes a system using a laser scan to detect cracks, pinholes, and thin regions in the shell.

Interior measurements involve breaking the egg to inspect its contents, and are typically done on eggs randomly chosen from large batches from a common source. Two interior measurements in common use are the measurement of yolk color using the Roche Yolk Color Fan, and the measurement of Haugh units. The Roche Yolk Color Fan has 15 sample colors, ranging from 1 (the lightest) to 15 (the darkest), which are visually matched to the color of the yolk.

Haugh units, which also characterize the freshness of an egg, were invented by Raymond Haugh in 1937, and are described by R. R. Haugh, “The Haugh unit for measuring egg quality,” U. S. Egg Poultry Magazine, No. 43, pages 552-555 and 572-573 (1937), as well as by USDA Egg-Grading Manual, Agricultural Handbook No. 75, Washington: USDA, July 2000, p. 34-35, and by William Stadelman and Owen J. Cotterill, eds., Egg Science and Technology, 4^{th} edition, New York: Food Products Press, 1995, pages 59-60. The egg is first weighed, then broken, and its contents are placed on a flat glass plate, with the egg temperature between 45 and 60 degrees F. (7 to 15 degrees C.). The height of a flat portion of the albumen is then measured using a micrometer. If the egg is very fresh, the albumen may not have a flat portion, in which case the height of the albumen is measured halfway between the yolk and the edge of the albumen. The Haugh units are then calculated using a formula involving the weight W in grams, and the height H in millimeters. Fresher eggs have albumen that does not spread out as much on the plate, and have a greater number of Haugh units. Eggs with more than 72 Haugh units are graded AA (if they also meet certain other criteria), eggs with between 71 and 60 Haugh units are graded A, eggs with between 59 and 31 Haugh units are graded B, and eggs with less than 30 Haugh units are graded C.

The traditional methods of manually and visually measuring egg interiors suffer from several problems. Skilled personnel are needed to make the measurements, and they take time. For both reasons, such measurements are expensive, and it may not be economical to make them. Haugh unit measurements are no longer in common use in the United States for this reason. Instead, qualitative observations of the yolk and albumen are commonly used for grading eggs. Haugh units are still widely used in Japan, where consumers are apparently more concerned about having scientific proof of egg freshness, and willing to pay higher prices for it. Measurements of yolk color with the Roche fan may give different results depending on the person making the measurements, due to differences in color vision among different people. The results may also vary depending on the lighting conditions, the size of the yolk, and the background color of the surface where the yolk is observed, even when an attempt is made to standardize these factors.

Deana R. Jones, “Proper Determination of Haugh Units,” National Egg Quality School Proceedings, pages 277-299 (May 19, 2003), states, in general terms, that “There is also more technologically advanced instrumentation” for measuring egg quality “which connect to a PC and allow for ‘real time’ results.”

The disclosures of all the references mentioned above are incorporated herein by reference.

### SUMMARY OF THE INVENTION

An aspect of some embodiments of the invention relates to an apparatus using a non-contact method for measuring the height of the albumen of a broken egg resting on a surface, such as a laser measurement or an ultrasound measurement. Optionally, the height measurement is made at a plurality of known locations, in order to obtain a single effective height parameter which can be used to find the Haugh units of the egg.

An aspect of some embodiments of the invention relates to an apparatus for measuring the height of the albumen of a broken egg, in which measurements are made at a plurality of different locations. Optionally the measurements are made automatically, without human intervention between the measurements. Optionally, the measurements are made without contacting the egg. Optionally, a scan is made over a region extending at least from the yolk to the edge of the albumen, when measuring the height. Optionally, the region extends to a portion of the edge of the albumen that is furthest from the yolk. Optionally, the results of the scan are used to find a substantial region where the albumen is relatively flat, and optionally the height in the flat region is obtained as a single effective height parameter which can be used to find the Haugh units. Optionally, the height parameter is found automatically by a processor, such as a computer, using the results of the height measurements.

Optionally, the egg is weighed, for example before it is broken, or after it is broken, and the weight of the egg and the height parameter are used as inputs for a processor which calculates the Haugh units.

An aspect of some embodiments of the invention relates to an apparatus and method for objectively measuring the color of the yolk of an egg by measuring the reflected light at one or more known wavelengths or distributions of wavelengths. Optionally, the color is calculated as an equivalent Roche Yolk Color Fan number. Optionally, the known distributions of wavelengths comprise distributions of wavelengths used in an RGB color measuring system. Optionally, the reflected light measurements are made automatically, and the results are then used as input for a processor which calculates the yolk color automatically.

An aspect of some embodiments of the invention relates to an apparatus and method for automatically determining a plurality of different parameters relating to egg quality, without the intervention of a human operator. Optionally, the parameters comprise one or both of Haugh units and yolk color. Optionally, the Haugh units and/or the yolk color are determined using one or more of the methods described.

There is thus provided, in accordance with an exemplary embodiment of the invention, a method of determining an effective height of albumen of an egg, for calculating Haugh units of the egg, the method comprising:
a) breaking the egg and placing its contents on a surface;
b) choosing at least one location at which to measure the height of the albumen; and
c) measuring the height of the albumen of the egg above the surface at the at least one location, using a non-contact measurement method.

Optionally, the height of the albumen is measured by a laser. Alternatively or additionally, the height of the albumen is measured by ultrasound.

In an embodiment of the invention, the method also includes producing at least one image of the albumen, and measuring the height of the albumen comprises analyzing the at least one image.

Optionally, choosing at least one location comprises analyzing the image to find a substantially flat region of the albumen.

There is further provided, in accordance with an exemplary embodiment of the invention, a method of finding Haugh units for an egg, the method comprising:
a) weighing the egg;
b) determining an effective height of the albumen of the egg, according to an embodiment of the invention; and
c) calculating the Haugh units for the egg, from the weight and the effective height of the albumen.

There is further provided, according to an exemplary embodiment of the invention, a method of determining an effective height of albumen of an egg, for calculating Haugh units of the egg, the method comprising:
a) breaking the egg and placing its contents on a surface;
b) choosing a plurality of different locations at which to measure the height of the albumen;
c) measuring the height of the albumen of the egg above the surface at the different locations; and

d) calculating an effective height of the albumen from the measured heights at the different locations.

Optionally, measuring the height of the albumen is done with a non-contact method.

Optionally, measuring the height at the different locations is done automatically, without human intervention between the measurements at the different locations. In an embodiment of the invention, calculating the effective height of the albumen comprises:
a) using the height measurements to locate a region of the surface where the albumen is substantially flat; and
b) using a height of the albumen in the substantially flat region as the effective height of the albumen.

Optionally, the different locations are at least approximately located on a line extending from the yolk to a point on the edge of the albumen where the albumen extends furthest from the yolk.

There is further provided, according to an exemplary embodiment of the invention, a method of finding Haugh units for an egg, the method comprising:
a) weighing the egg;
b) determining an effective height of the albumen of the egg, according to an embodiment of the invention; and
c) calculating the Haugh units for the egg, from the weight and the effective height of the albumen.

Optionally, determining the effective height of the albumen is done after weighing the egg.

There if further provided, according to an exemplary embodiment of the invention, a method of objectively determining the yolk color of an egg, the method comprising:
a) illuminating the yolk with light of a first illuminating distribution of wavelengths;
b) measuring the intensity of the light reflected from the yolk, using a detector with a first response distribution as a function of wavelength;
c) calculating a first reflection coefficient of the yolk for a first distribution of wavelengths depending on the first illuminating distribution and the first response distribution, using the measured intensity of reflected light; and
d) using at least said first reflection coefficient to find the yolk color of the egg.

Optionally, the method also includes:
a) illuminating the yolk with light of a second distribution of wavelengths and measuring the intensity of the light reflected from the yolk using a detector with a second response distribution, wherein either the second illuminating distribution is different from the first illuminating distribution, or the second response distribution is different from the first response distribution, or both;
b) calculating the reflection coefficient of the yolk for a second distribution of wavelengths depending on the second illuminating distribution and the second response distribution, and different from the first distribution of wavelengths, using the measured intensity of reflected light; and
c) using at least said first and second reflection coefficients to find the yolk color of the egg.

Optionally, the method also includes:
a) illuminating the yolk with light of a third distribution of wavelengths and measuring the intensity of the light reflected from the yolk using a detector with a third response distribution;
b) calculating the reflection coefficient of the yolk for a third distribution of wavelengths depending on the third illuminating distribution and the third response distribution, and different from both the first and second distributions of wavelengths, using the measured intensity of reflected light; and
c) using at least said first, second, and third reflection coefficients to find the yolk color of the egg.

Optionally, the first, second, and third distributions of wavelengths appear red, green, and blue to the human eye when adapted to daylight.

Optionally, finding the color of the egg comprises finding a Roche Yolk Color Fan number corresponding to the color of the egg.

There is further provided, according to an exemplary embodiment of the invention, a method of testing egg quality, comprising automatically measuring a first parameter and a second parameter, without any human intervention between measuring the first parameter and the second parameter.

Optionally, one of the parameters is an effective height of albumen for measuring Haugh units. Optionally, the effective height of albumen is measured using a method according to an embodiment of the invention.

Optionally, one of the parameters is yolk color. Optionally, another one of the parameters is an effective height of the albumen for calculating Haugh units. Optionally, the yolk color is measured using a method according to an embodiment of the invention.

There if further provided, according to an exemplary embodiment of the invention, apparatus for measuring egg quality, comprising:
a) a plate for holding the albumen and the yolk of the egg after it is broken; and
b) a non-contact height sensor adapted for measuring the height of the albumen at at least one location on the plate.

Optionally, the non-contact height sensor comprises a laser and a light detector. Optionally, the non-contact height sensor comprises an ultrasound source and an ultrasound detector.

Optionally, the non-contact height sensor comprises at least one camera adapted to produce at least one image of the albumen, and an image analyzer adapted to analyze the at least one image to find the height of the albumen at the at least one location. Optionally, the at least one camera is adapted to include the yolk in the at least one image, and the image analyzer is adapted to analyze the at least one image to find the color of the yolk.

In an embodiment of the invention, the apparatus includes:
a) a scale for weighing the egg; and
b) a controller adapted to use the weight of the egg and results of measuring the height of the albumen at at least one location to calculate the Haugh units of the egg.

There is further provided, according to an exemplary embodiment of the invention, apparatus for measuring egg quality, comprising:
a) a plate for holding the albumen and the yolk of the egg after it is broken;
b) a height sensor adapted for measuring the height of the albumen at a plurality of locations on the plate; and
c) a controller adapted to direct the height sensor to measure the height of the albumen at a specified set of different locations, and to receive results of the measurements.

Optionally, the height sensor is a non-contact height sensor.

Optionally, the controller is adapted to find an effective height of the albumen for calculating Haugh units, using the results of the measurements of height at the different locations.

Optionally, the apparatus also includes a scale for weighing the egg which outputs the weight to the controller, and the controller is adapted to use the weight of the egg and the effective height of the albumen to calculate the Haugh units of the egg.

There if further provided, in accordance with an exemplary embodiment of the invention, apparatus for measuring egg quality by finding yolk color, comprising:
a) a plate for holding the albumen and the yolk of the egg after it is broken;
b) one or more light sources comprising at least a first light source, all the light sources positioned and oriented to illuminate the yolk when it is on the plate, with light of a first illuminating distribution of wavelengths;
c) one or more light detectors comprising at least a first light detector, with a first response distribution, all the light detectors positioned and oriented to measure the intensity of light which reflects from the yolk; and
d) a controller adapted to calculate a first reflection coefficient of the yolk for a first distribution of wavelengths depending on the first illuminating distribution and the first response distribution, using the measured intensity of the reflected light, and to use at least the first reflection coefficient to find the yolk color.

Optionally, the light sources comprise at least a second light source with a second illuminating distribution of wavelengths, or the light detectors comprise at least a second light detector with a second response distribution, or both, wherein the controller is adapted to calculate a second reflection coefficient of the yolk for a second distribution of wavelengths, differing from the first distribution of wavelengths because it depends on a different one of the illuminating distributions, or a different one of the response distributions, or both, and to use at least the first and second reflection coefficients to find the yolk color.

Optionally, the light sources and light detectors jointly comprise at least three different combinations of light source illuminating distributions and light detector response distributions, wherein the controller is adapted to calculate a third reflection coefficient of the yolk for a third distribution of wavelengths, different from the first and second distributions of wavelengths because it depends on a different one of the illuminating distributions, or a different one of the response distributions, or both, and to use at least the first, second, and third reflection coefficients to find the yolk color.

Optionally, the apparatus includes:
a) a non-contact height sensor adapted to measure the height of the albumen at a plurality of different locations on the plate and to output the results of the height measurements to the controller; and
b) a scale for weighing the egg which outputs the weight to the controller;
   and the controller is adapted to find an effective albumen height from the height measurements at the different locations, and to calculate Haugh units of the egg from the effective height of the albumen and the weight of the egg.

### BRIEF DESCRIPTION OF THE DRAWINGS

Exemplary non-limiting embodiments of the invention are described in the following sections with reference to the drawings. The drawings are generally not to scale and the same or similar reference numbers are used for the same or related features on different drawings.

FIG. 1 is a flowchart for a method of determining egg quality, according to an exemplary embodiment of the invention;

FIG. 2 schematically shows an external perspective view of an apparatus for measuring egg quality, using the method of FIG. 1;

FIG. 3 schematically shows a side cross-sectional view of the apparatus shown in FIG. 2;

FIG. 4 schematically shows a top view of the contents of an egg on a plate in the apparatus shown in FIGS. 2 and 3;

FIG. 5A schematically shows a front or rear cross-sectional view of a part of an apparatus for measuring egg quality, according to another exemplary embodiment of the invention;

FIG. 5B schematically shows a front, rear, or side cross-sectional view of a part of an apparatus for measuring egg quality, according to another exemplary embodiment of the invention;

FIG. 6A schematically shows a side cross-sectional view of a part of the apparatus shown in FIGS. 2 and 3; and

FIG. 6B schematically shows a side cross-sectional view of a part of an apparatus for measuring egg quality, according to another exemplary embodiment of the invention.

### DETAILED DESCRIPTION OF EXEMPLARY EMBODIMENTS

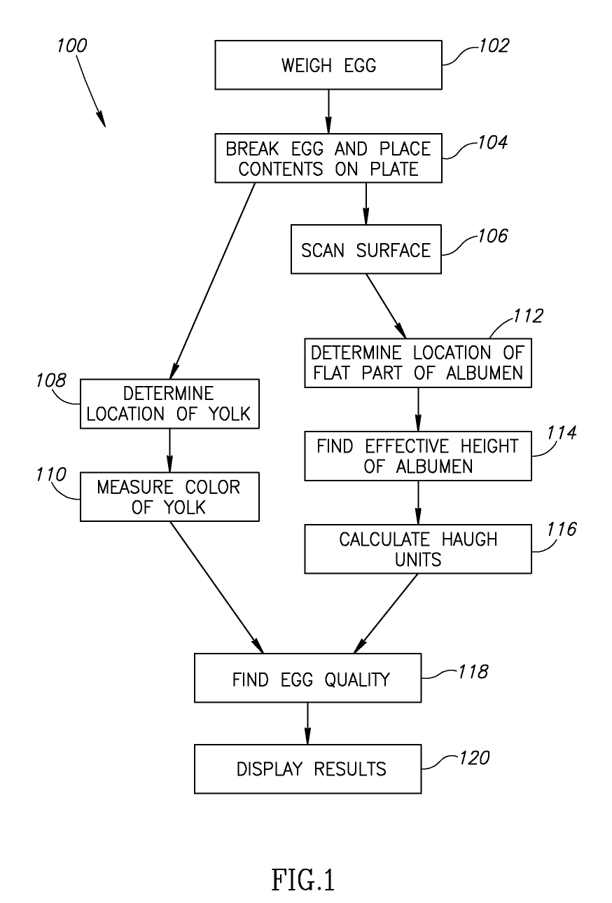

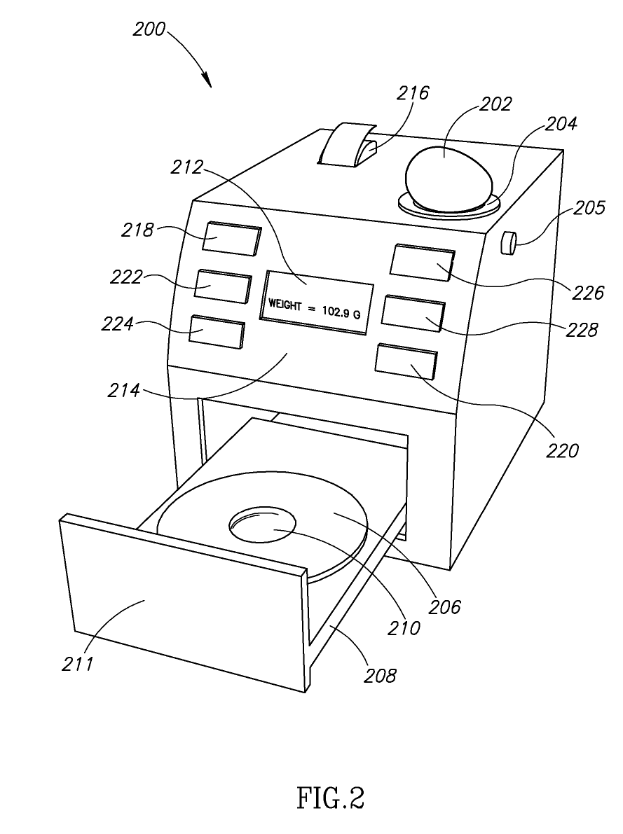

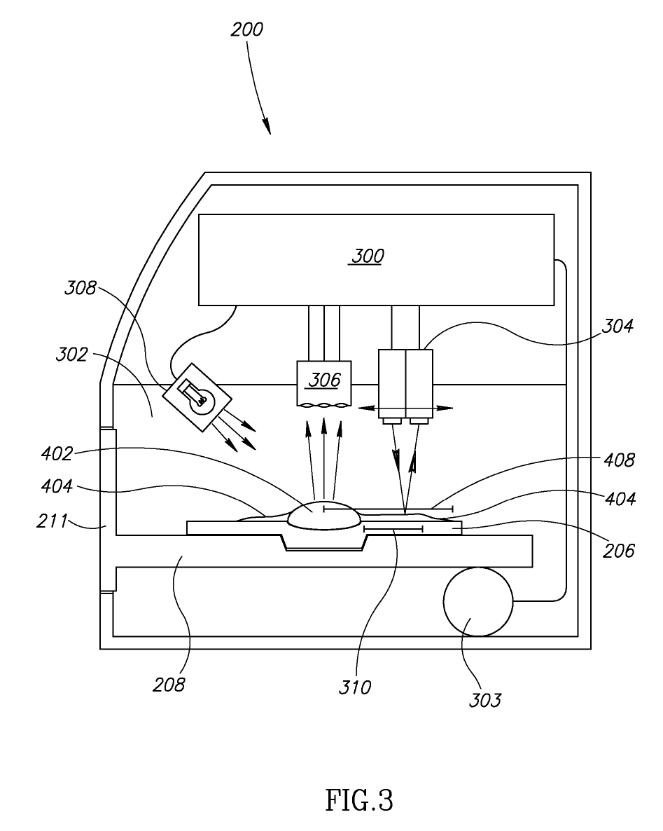

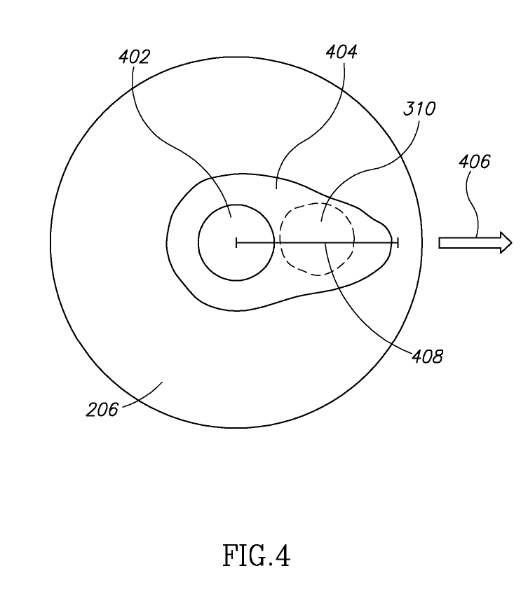

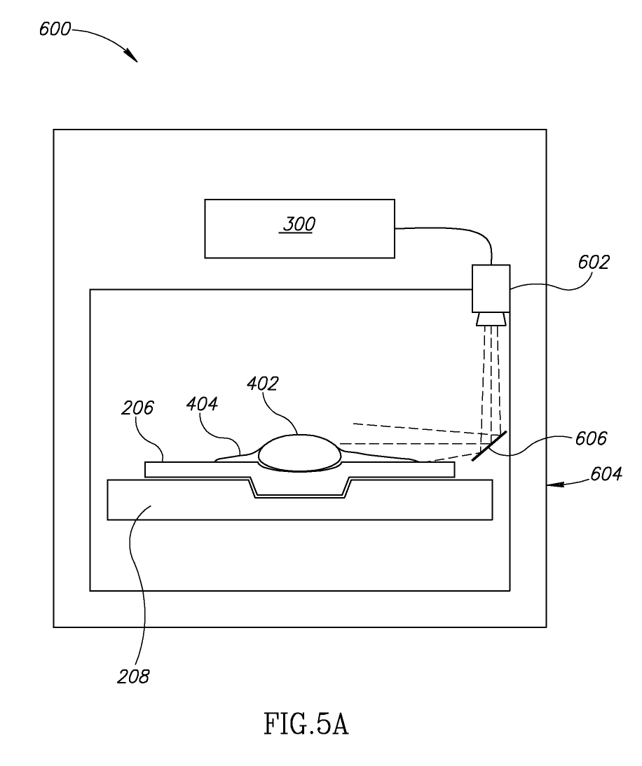

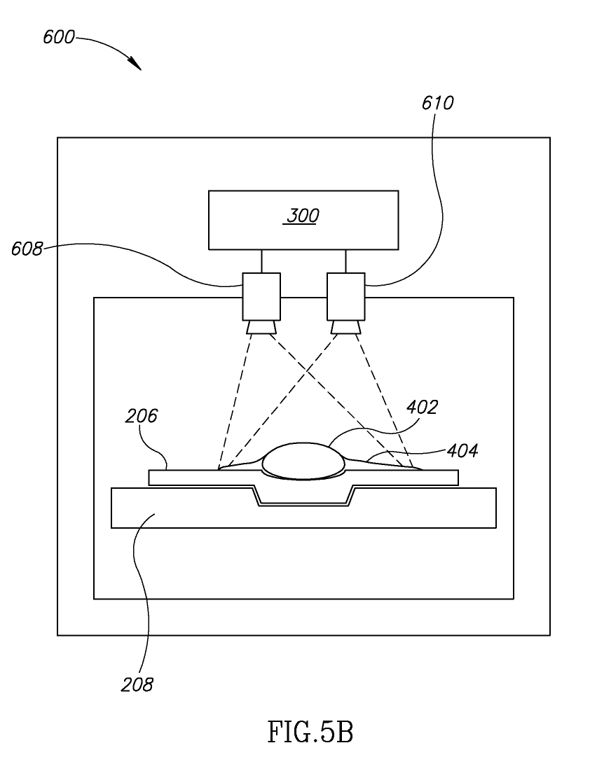

FIG. 1 shows a flowchart 100, for a method of determining egg quality according to an exemplary embodiment of the invention. The acts shown in flowchart 100 will be described with reference to an egg-testing apparatus 200 shown in FIGS. 2 and 3, and to parts of apparatus 200 shown in FIGS. 4, 5A, and 5B. Optionally, apparatus 200 has dimensions of between 250 mm and 350 mm in length, width, and height, and weighs between 5 and 8 kg.

At 102, an egg is optionally weighed. Optionally, an unbroken egg, such as egg 202 shown in FIG. 2, is weighed on a scale 204 built into apparatus 200, for example on the top surface of apparatus 200 as shown in FIG. 2, or inside apparatus 200. Scale 204 comprises, for example, a load cell, with a digital output. Scale 204 has a range, for example, of 20 grams to 150 grams, with a precision of 0.1 gram. Optionally, scale 204 can hold a weight of at least 300 grams without being damaged. Optionally, the weight of the egg is recorded in a memory, for example in the memory of a microprocessor 300, shown in FIG. 3, built into apparatus 200. The weight is then available for use later in calculations pertinent to egg quality, for example for calculation of Haugh units as will be described below, without any need for a human operator to record the weight and input it later. Optionally, scale 204 has a locking mechanism 205, which prevents damage to scale 204 when apparatus 200 is moved, for example.

At 104, the egg is broken, and its interior contents (yolk and albumen) are placed on a plate, for example a plate 206, mounted on a tray 208 built into apparatus 200. Optionally, tray 208 opens automatically, under the control of microprocessor 300, after the egg is weighed, and the user is prompted to break the egg and place its contents on plate 206. Optionally, this is done when the temperature of the egg is between 45 and 60 degrees Fahrenheit, in accordance with the usual standard for determining Haugh units. Optionally, plate 206 is flat, at least over most of its area, which has the potential advantage that using a flat plate may facilitate making an accurate measurement of the effective height of the albumen. Optionally, plate 206 has a relatively shallow depression 210, for example at its center, into which the yolk is placed. Depression 210 has the potential advantage that it keeps the yolk at a known position, facilitating the measurement of yolk color. Optionally, care is taken not to have any of the albumen on top of the yolk, since this can make the yolk color measurement and possibly the albumen height measurement inaccurate. Optionally, eggs are not tested if they have two yolks, since this may make it difficult to position the yolks so that the yolk color and the height of the albumen can be measured accurately.

Plate 206 is optionally mounted on tray 208 in a manner that allows plate 206 to rotate relative to tray 208. After the contents of the egg are placed on plate 206, plate 206 is optionally rotated so that the albumen extends furthest from the yolk in a particular direction relative to the tray, for example in a direction toward the back of tray 208. FIG. 4 is a top view of an egg resting on plate 206, with a yolk 402 at the center of plate 206, and albumen 404 extending furthest in a direction, indicated by arrow 406, toward the back of tray 208. Orienting the contents of the egg in a known direction has the potential advantage that it may facilitate measuring the height of the albumen.

Optionally, any bubbles, or the chalaza (the band of tissue connecting the yolk to the membrane on the inside of the shell), or bloodspots on the yolk, are removed from the surface of the egg contents, after the egg contents are placed on plate 206. Optionally, they are at least moved away from a part of the surface of the albumen where the height is measured, and from a part of the surface of the yolk where the color is measured. Where these parts of these surfaces may be located is described below. Optionally, data from points which are recognized by data analysis software as having a bubble or the chalaza or a bloodspot is ignored, in measuring the height of the albumen and/or the color of the yolk. For example, data is ignored if it is very different from data from a nearby point, or from nearby points on two sides.

Optionally, tray 208, together with plate 206, is slid into apparatus 200, once the interior contents of the egg have been placed on plate 206. Measurements of the contents of the egg are then optionally made in an enclosure 302 inside apparatus 200, shown in FIG. 3. Optionally, a motor 303, shown in FIG. 3, causes tray 208 to slide into and out of apparatus 200. Alternatively or additionally, tray 208 slides manually. Optionally, there is a partition 211 at the outer end of tray 208, which blocks outside light from entering enclosure 302 while measurements are being made.

At 106, a scan is optionally made of the contents of the egg on plate 206, measuring its height as function of position on plate 206, for example as a function of position along a line segment 408 extending from yolk 402 past the furthest extent of albumen 404, which is, for example, 10 cm long. It should be noted that for purposes of measuring Haugh units, the height of the albumen is defined as the height of a flat region of the albumen, if such a region exists. The flat region is generally measured on the side of the yolk where the albumen extends furthest from the yolk. Optionally the height of the albumen is measured at points spaced at intervals of 1 mm or 0.5 mm or 0.2 mm or 0.1 mm. Optionally, the height at each point is measured to within 0.5 mm or 0.25 mm or 0.1 mm, optionally over a range of 0 to 30 mm.

The scan is made by a scanner 304, located in enclosure 302. Optionally, scanner 304 is a scanner that uses sound waves, for example an ultrasound scanner. A suitable ultrasound scanner is, for example, the MassaSonic™ M-5000/220 Smart Ultrasonic Sensor, sold by Vydas International Marketing, in Passfield, Hants, UK, and described in a brochure and data sheet [retrieved Jul. 10, 2006], retrieved from the Internet <URL: http://www.vydas.co.uk/prod13.htm> and <URL: http://www.vydas.co.uk/PDF/MP/M5000220DataSheet.pdf>.

Alternatively or additionally, scanner 304 is a scanner that uses light, for example a laser scanner, or is any other type of scanner known in the art that is suitable for measuring the height of albumen and yolk resting on a surface. Optionally, data on the height as a function of position on plate 206 is recorded in a memory, for example the memory of microprocessor 300.

In some embodiments of the invention, the height of the albumen as a function of position on plate 206 is determined by producing one or more images of albumen 404, and optionally also yolk 402, on plate 206, and analyzing the images. FIGS. 5A and 5B show two exemplary ways of producing the images in an apparatus 600, similar to apparatus 200 in FIGS. 2 and 3. In FIG. 5A, a camera 602, for example a digital camera, produces an image of albumen 404, and optionally yolk 402, as seen from a side 604 of plate 206. FIG. 5A shows camera 602 located above plate 206, and pointed toward a mirror 606 oriented at a 45 degree angle so that camera 602 views albumen 404 and yolk 402 from side 604. Alternatively there is no mirror 606, and camera 602 is located to the side of plate 206, for example at the location where mirror 606 is shown in FIG. 5A, and is pointed directly toward albumen 404 and yolk 402. The arrangement shown in FIG. 5A has the potential advantage that camera 602 is not as close to albumen 404 and yolk 402, and need not have such a large field of view and depth of field. In either case, camera 602 produces a profile image of the egg that resembles the profile of the egg shown in FIG. 3. The profile image is analyzed, for example by microprocessor 300, to produce data on the height of the albumen as a function of position on plate 206, similar to the data produced by scanner 304 in the embodiment of the invention shown in FIG. 3.

FIG. 5B shows a different embodiment of the invention for obtaining data on the height of the albumen as a function of position on plate 206, from images. Two cameras 608 and 610, or a single stereoscopic camera, produce images of albumen 404, and optionally of yolk 402, from different points of view. Alternatively, a single camera moves into two different positions, and produces images from each point of view in succession. The camera positions may be nearly above the egg, for example, or at a large oblique angle to the egg. The resulting stereoscopic images are analyzed to produce data on the height of the albumen as a function of position on plate 206. Optionally, a pattern of light is projected onto the surface of the albumen, for example a raster or grid pattern, while the images are made, which has the potential advantage that it may simplify the analysis of the stereoscopic images. In some embodiments of the invention, data on the height of the albumen as a function of position on plate 206 is obtained by analyzing a single image with a pattern projected on it. Optionally, plate 206 is black in color, which has the potential advantage of reducing the brightness of light from the pattern which travels through the transparent albumen and reflects from the surface of the plate, relative to the light that reflects from the surface of the albumen.

If the results of the scan or the imaging appear to indicate that something is wrong, for example plate 206 is oriented in a wrong direction, or the egg has two yolks, or there is no egg on plate 206 at all, then an error message is optionally displayed, for example under the control of microprocessor 300. Tray 208 optionally opens automatically, and the user is optionally instructed to make sure that the egg is present on plate 206, with the furthest extent of the albumen oriented in the right direction, and with only one yolk.

At 108, the location of the yolk is determined. Optionally, the yolk is assumed to be at the location of depression 210, for example at the center of plate 206, where the user is instructed to place the yolk. If an attempt to measure the color of the yolk at this location does not give reasonable results, then an error message is optionally displayed, under the control of microprocessor 300 for example. Tray 208 optionally opens automatically, and the user is instructed to make sure the yolk is located at the expected place.

Alternatively, the location of the yolk is determined by the result of a scan, for example a two-dimensional scan over an area of plate 206. Optionally, the scan comprises a scan of the height of the contents of the egg above the plate, using scanner 304. For example, the highest point is identified as the center of the yolk, and a circular region around the highest point, where the surface is convex, is identified as the rest of the yolk. Additionally or alternatively, the scan is a scan of the color of the surface, and a yellow region, approximately circular and of the right size range, is identified as the yolk. Optionally, the boundaries of the yolk are not identified, but only one point, or a limited number of points, are identified as part of the yolk.

At 110, a measurement is made of the color of the yolk. This is done, for example, by a color sensor 306, and a light source 308, located in enclosure 302. Optionally, light source 308 illuminates the yolk from an angle approximately 45 degrees from vertical, while color sensor 306 is located approximately directly above the yolk. This arrangement has the potential advantage that a relatively large amount of the light is reflected diffusely into color sensor 306, but relatively little light is reflected specularly into color sensor 306. The color sensor is optionally an RGB color sensor, which detects light from light source 308 of three different wavelengths, or three different distributions of wavelength, reflected from the surface of the yolk, and measures the reflection coefficient of each wavelength or distribution. Optionally, light source 308 is a white light source, for example a halogen lamp. The measurement results are corrected for the actual spectrum of the white light source.

Alternatively, color sensor 306 comprises a single sensor, sensitive to a broad range of wavelengths, and light source sequentially produces light of three different wavelengths, or wavelength distributions, while color sensor 306 measures the reflection coefficient of each one.

Optionally, the wavelengths or distributions correspond at least approximately to the peaks of sensitivity, or distributions of sensitivity, of the red, green, and blue sensitive cones of the human retina. The color detected by the color sensor is optionally matched to a closest color on the Roche Yolk Color Fan, so that the number corresponding to the yolk color is found to the nearest integer, from 1 to 15.

Optionally, the number corresponding to the yolk color is found to the nearest tenth of an integer. Optionally, the reflectivity is measured at fewer than three wavelengths or distributions, but the results are optionally matched to a color on the Roche Yolk Color Fan by using information about the normal range of color of egg yolks. Optionally, the color is measured at a plurality of points on the surface of the yolk, or over an extended area of the surface of the yolk, and the results are averaged, in order to improve accuracy for example. Optionally, the color is measured at only a single point identified as belonging to the yolk. Optionally, the result of the color measurement, for example a corresponding number on the Roche Yolk Color Fan, is stored in the memory of microprocessor 300.

In some embodiments of the invention, instead of or in addition to using color sensor 306 to measure the color of the yolk, one or more cameras, such as cameras 608 and 610 in FIG. 5B, are used to produce one or more images of the egg, including the yolk. The images are then analyzed to identify the yolk and to determine the color of the yolk. This method may be especially convenient to use in embodiments of the invention such as those shown in FIG. 5A or FIG. 5B, where there are already one or more cameras, and image analysis software, used to measure the height of the albumen. In some embodiments of the invention, cameras and image analysis software are used only for measuring the color of the yolk.

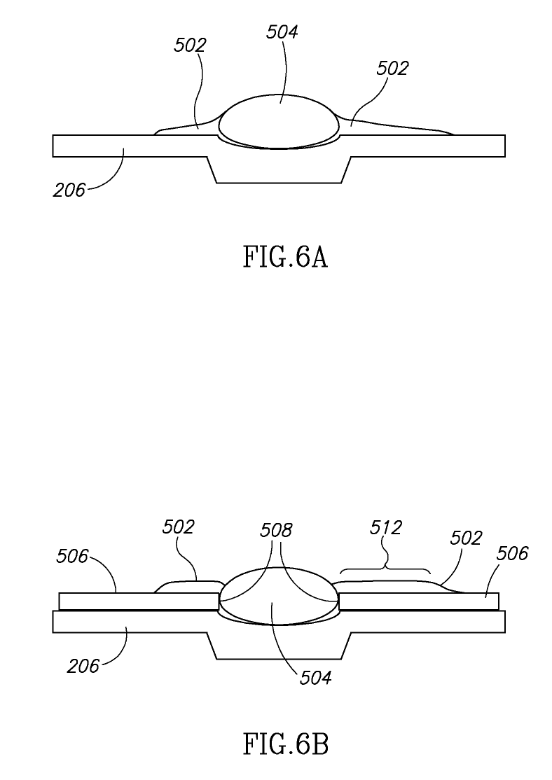

At 112, the data on height as a function of position on the plate 206 is used to look for a region 310 where the height of the albumen is relatively flat. This region is optionally used, at 114, to find an effective height of the albumen, for purposes of calculating the Haugh units. For example, the height of the albumen at one point in the flat region is used as the effective height, or an average of the height at a plurality of points in the flat region is used. In very fresh eggs, there may not be such a flat region, since the albumen may be so viscous that it does not flow very far from the yolk. This situation is shown in FIG. 6A, where the height of albumen 502 monotonically decreases going away from yolk 504, with no flat region. In this case, an adapter 506, shown in FIG. 6B, is optionally used to produce a flat region of the albumen. Adapter 506, which is shown in side cross-section in FIG. 6B, comprises a circular disk, covering plate 206, with a hole 508 in its center, approximately the size of the yolk, and which optionally fits over depression 210, effectively making the depression deeper. When adapter 506 is placed over plate 206, and yolk 504 is placed in hole 502, then yolk 504 sinks lower down, relative to albumen 502, than it would if adapter 506 were not used, as may be seen by comparing FIG. 6B, with adapter 506 present, with FIG. 6A, with no adapter. As a result, albumen 502 has a flat region 512 in FIG. 6B.

Alternatively, if there is no flat region of the albumen, then the effective height of the albumen is not found, and the Haugh units are not calculated, but when an overall indication of egg quality is found (see below, in the description of 118), the egg is rated as being very fresh, for example grade AA.

Alternatively, a different method is used to find the effective height of the albumen. The method used is, for example, a method consistent with the definition of effective height described in the USDA Egg-Grading Manual, referenced above, for very fresh eggs in which there is no flat region in the albumen. In such a method, a point on the edge of the yolk is found on line segment 408, for example by using data on the height to find an inflection point, or a point of maximum negative curvature, in the surface of the contents of the egg. The edge of the albumen is found, for example by finding the point on line segment 408 where the height of the egg contents goes to zero. The effective height of the albumen is then optionally found by using the height at a point halfway between the boundary of the yolk and the boundary of the albumen.

A potential advantage of using this method, rather than the method shown in FIG. 6B and described above, is that this method is accepted by the US Department of Agriculture as a way to determine Haugh units when there is no flat region of the albumen. The method shown in FIG. 6B might not give the same result for the same egg. Optionally, the method shown in FIG. 6B is used, but a correction is made to the measured height of the albumen, in order to make the results consistent with the method described in the USDA Egg-Grading Manual.

Optionally, once the yolk color and the effective height of the albumen are measured, tray 208 automatically slides open, under the control of microprocessor 300 for example. Plate 206 may then be removed from tray 208, and cleaned off, before the next egg is tested.

At 116, the weight of the egg and the effective height of the albumen are optionally used to calculate the Haugh units for the egg. Optionally, the calculation is done by microprocessor 300, for example using the values of weight and height stored in memory. The algorithm for calculating the Haugh units is, for example, a standard algorithm shown in one of the references for Haugh units listed above. For example, the number of Haugh units is equal to
100 log(H−1.7W^{0.37}+7.6)
where H is the height of the albumen in millimeters, and W is the weight of the egg in grams.

In some embodiments of the invention, instead of weighing the egg on scale 204 before the egg is broken, there is a scale built into plate 206, and the egg is weighed sometime after it is broken and its contents are placed on plate 206. Optionally, in this case, the formula for Haugh units is optionally modified, to take into account that the weight measured does not include the weight of the shell. Alternatively, the broken shell is also placed on plate 206, for example to a side of plate 206 where it will not interfere with the measurements of albumen height and yolk color, and the shell is included in the weight. Weighing the egg while it is on plate 206 has the potential advantage that it could save time, since it eliminates the need for a user to place the egg on the scale and to wait for the weight to register.

At 118, an overall indication of egg quality is optionally found, using the Haugh units and/or the yolk color. Optionally, other information is also used in finding the indication of egg quality, for example, the results of an external inspection of the egg, such as that described in U.S. Pat. No. 5,615,777 to Weichman. Optionally, the overall indication of egg quality is expressed as a standard egg grade, such as AA, A, B or C. Optionally, eggs with at least 72 Haugh units are graded AA, eggs with between 60 and 71 Haugh units are graded A, eggs with between 31 and 59 Haugh units are graded B, and eggs with less than 31 Haugh units are graded C. Optionally, eggs must meet additional requirements in order to be graded AA, or in order to be graded A.

At 120, any of the results found, for example the weight, the effective height of the albumen, the Roche Fan number for the yolk color, the Haugh units, and the egg grade, are displayed on a display screen 212, for example a liquid crystal display screen, on a control panel 214, either together or separately. Display screen 212 has, for example, four lines, which can display up to 20 characters each. Optionally, any of these results are printed out by a printer 216 in apparatus 200. Optionally, control panel 214 also includes one or more buttons used by a user to initiate the egg-testing procedure, and to otherwise control the procedure. For example, control panel 214 may include one or more of a start button 218 to start the procedure; a tray control button 220 to open and close tray 208; a weight display button 222 to display the latest weight on display screen 212; a results display button 224 to display all the results; a memory button 226 to display the results of previous tests that are stored in the memory of microprocessor 300; and a print button 228 to print out the results shown on display screen 212, or to print an extra copy of the results. Optionally, results for up to 100 previous tests are stored in the memory of microprocessor 300. Optionally, microprocessor 300 calculates an average value, and optionally a standard deviation as well, of the results for one or more of the types of measurement, for the tests whose results are stored in memory. Optionally, outliers are removed, before calculating the average value and standard deviation. Optionally, the memory of microprocessor 300 is volatile, with all stored results erased when power to apparatus 200 is turned off. Optionally, results are printed immediately after each test, so that data is not lost if apparatus 200 accidentally loses power. It is also noted that, as described below, data from each test is optionally stored in an external computer, immediately after each test. Alternatively, the memory of microprocessor 300 is non-volatile.

Optionally, display screen 212 is also used, or a different output device is used, to give instructions to a user of apparatus 200. For example, after weighing the egg, the user is instructed to break the egg and place its contents on plate 206, and to close tray 208, making it slide into enclosure 302, for example by pushing tray control button 220. If apparatus 200 is unable to determine a flat region of the albumen, or unable to measure the color of the yolk, because the yolk is in the wrong position on plate 206, or because plate 206 is not turned so that the albumen extends in the right direction, then tray 208 optionally opens, and display screen 212 optionally instructs the user to correct the problem, and to close the tray again.

Optionally, apparatus 200 is connected to a computer by a communications link, such as an RS-232 cable, which connects to apparatus 200 at the back, for example. Any of the functions described above as performed by microprocessor 300 are optionally performed instead by the computer. For example, test results are optionally stored in the memory of the computer rather than in the memory of microprocessor 300. The test procedure is optionally under the control of the computer, and a user interface, optionally emulating control panel 214, is optionally displayed on a screen of the computer. A printer controlled by the computer is optionally used to print out the results, optionally on a full sheet of paper, rather than printer 216, which optionally uses a narrow roll of paper.

The invention has been described in the context of the best mode for carrying it out. It should be understood that not all features shown in the drawings or described in the associated text may be present in an actual device, in accordance with some embodiments of the invention. Furthermore, variations on the method and apparatus shown are included within the scope of the invention, which is limited only by the claims. Also, features of one embodiment may be provided in conjunction with features of a different embodiment of the invention. As used herein, the terms “have”, “include” and “comprise” or their conjugates mean “including but not limited to.”

1. A method of determining an effective height of albumen of an egg, for calculating Haugh units of the egg, the method comprising:
a) breaking the egg and placing its contents on a surface;
b) choosing at least one location at which to measure the height of the albumen; and
c) measuring the height of the albumen of the egg above the surface at the at least one location, using a non-contact measurement method.

2. A method according to claim 1, wherein the height of the albumen is measured by a laser.

3. A method according to claim 1, wherein the height of the albumen is measured by ultrasound.

4. A method according to claim 1, also including producing at least one image of the albumen, wherein measuring the height of the albumen comprises analyzing the at least one image.

5. A method according to claim 4, wherein choosing at least one location comprises analyzing the image to find a substantially flat region of the albumen.

6. A method of finding Haugh units for an egg, the method comprising:
a) weighing the egg;
b) determining an effective height of the albumen of the egg, according to the method of claim 1; and
c) calculating the Haugh units for the egg, from the weight and the effective height of the albumen.

7. A method of determining an effective height of albumen of an egg, for calculating Haugh units of the egg, the method comprising:
a) breaking the egg and placing its contents on a surface;
b) choosing a plurality of different locations at which to measure the height of the albumen;
c) measuring the height of the albumen of the egg above the surface at the different locations; and
d) calculating an effective height of the albumen from the measured heights at the different locations.

8. A method according to claim 7, wherein measuring the height of the albumen is done with a non-contact method.

9. A method according to claim 7, wherein calculating the effective height of the albumen comprises:
a) using the height measurements to locate a region of the surface where the albumen is substantially flat; and
b) using a height of the albumen in the substantially flat region as the effective height of the albumen.

10. A method according to claim 7, wherein the different locations are at least approximately located on a line extending from the yolk of the egg to a point on the edge of the albumen where the albumen extends furthest from the yolk.

11. A method of finding Haugh units for an egg, the method comprising:
a) weighing the egg;
b) determining an effective height of the albumen of the egg, according to the method of claim 7; and
c) calculating the Haugh units for the egg, from the weight and the effective height of the albumen.

12. Apparatus for measuring egg quality, comprising:
a) a plate for holding the albumen and the yolk of the egg after it is broken; and
b) a non-contact height sensor adapted for measuring the height of the albumen at at least one location on the plate.

13. Apparatus according to claim 12, wherein the non-contact height sensor comprises a laser and a light detector.

14. Apparatus according to claim 12, wherein the non-contact height sensor comprises an ultrasound source and an ultrasound detector.

15. Apparatus according to claim 12, wherein the non-contact height sensor comprises at least one camera adapted to produce at least one image of the albumen, and an image analyzer adapted to analyze the at least one image to find the height of the albumen at the at least one location.

16. Apparatus according to claim 12, including:
a) a scale for weighing the egg; and
b) a controller adapted to use the weight of the egg and results of measuring the height of the albumen at at least one location to calculate the Haugh units of the egg.

17. Apparatus for measuring egg quality, comprising:
a) a plate for holding the albumen and the yolk of the egg after it is broken;
b) a height sensor adapted for measuring the height of the albumen at a plurality of locations on the plate; and
c) a controller adapted to direct the height sensor to measure the height of the albumen at a specified set of different locations, and to receive results of the measurements.

18. Apparatus according to claim 17, wherein the height sensor is a non-contact height sensor.

19. Apparatus according to claim 17, wherein the controller is adapted to find an effective height of the albumen for calculating Haugh units, using the results of the measurements of height at the different locations.

20. Apparatus according to claim 19, also including a scale for weighing the egg which outputs the weight to the controller, wherein the controller is adapted to use the weight of the egg and the effective height of the albumen to calculate the Haugh units of the egg.
